(19) United States
(12) Patent Application Publication
(10) Pub. No.: US 2011/0196236 A1
(43) Pub. Date: Aug. 11, 2011

(54) SYSTEM AND METHOD OF AUTOMATED GESTATIONAL AGE ASSESSMENT OF FETUS

(76) Inventors: Gokul Swamy, Bangalore (IN); Prateep Mukherjee, Hyderabad (IN); Jyotirmoy Banerjee, Bangalore (IN); Sheshadri Thiruvenkadam, Bangalore (IN)

(21) Appl. No.: 12/701,755

(22) Filed: Feb. 8, 2010

(51) Int. Cl. A61B 8/14

(52) U.S. Cl. 600/443

(57) ABSTRACT

An ultrasound system includes a transducer array comprising a multiplicity of transducer elements configured to acquire image data of an object, a display system for displaying an image of the object based on the acquired image data, and an image processor module. The image processor module is programmed to calculate the curvature of the image, and identify an object feature based on the calculated curvature and based on known feature tendencies of the object.

### BACKGROUND OF THE INVENTION

Embodiments of the invention relate generally to ultrasound imaging and, more particularly, to an apparatus and method of automatically assessing human gestational age.

As is well known, in ultrasound imaging a series of high-frequency sonic pulses is generated, and these pulses “bounce” or echo off of various objects in their path. Specifically, different structures in a patient's body exhibit different levels of impedance, and ultrasound echoes are generated when the ultrasound signals contact impedance boundaries between these structures. An interval between the emission of the pulses and the receipt of the corresponding echoes is measured to determine the distance between the source of the pulse and the impedance boundary from which the echo resulted. In addition, the relative intensity of the echo conveys information regarding the nature of the tissues causing the echoes. Different tissues exhibit different levels of impedance to the ultrasound signals. Therefore, varying impedance differentials exist, for example, at the boundary between muscle tissue and bone as opposed to the boundary between fatty tissue and organ tissue. As a result, when an ultrasound strikes the impedance boundary between muscle tissue and bone, a more robust echo is generated than the echo generated when an ultrasound pulse strikes the impedance boundary between fatty tissue and organ tissue. Ultimately, it is the mosaic assembled from each of these echoes received, reflecting the position and the nature of the objects causing the echoes, that constitutes the multi-dimensional images obtained through the use of ultrasound imaging.

Typically, ultrasound images are routinely used to assess fetal growth and determine or predict gestational age (GA) of a fetus. Ultrasound measurements of specific features of fetal anatomy such as the head, abdomen or the femur from 2-D or 3-D image data are used in the determination of GA, assessment of growth patterns and identification of anomalies.

In one example, measurement of femur length is a significant indicator of fetal growth in the second and third trimesters of pregnancy. In common clinical practice, the ultrasound transducer is moved over the abdomen until the femur is visible in a standard scan plane in which the bone surface is approximately normal to the ultrasound beam. The length of the femur is then measured by indicating its endpoints on the visual display with a mouse-like mechanism incorporated into the image display station. The GA corresponding to the measurement is read off of standard Obstetric (OB) Tables. Typically, femur length measurement involves manual measurement by a trained ultrasonographer.

In another example, fetal head circumference is also an indicator of GA and can also be used to gauge abnormalities in the fetal growth pattern. Typically, fetal head circumference measurement also involves manual measurement by a trained ultrasonographer.

Fetal ultrasound images are invariably contaminated by a number of factors that can compromise a diagnosis. The factors include but are not limited to near field haze due to a fatty layer in the abdomen, unpredictable movement, limb placement of the fetus, and ubiquitous speckle noise. Operator variability also limits reproducibility of ultrasound imagery and measurement. Early efforts at improving robustness and accuracy of clinical workflow have tended to focus on semi-automated methods that include, for example, femur segmentation. The semi-automated methods include approaches such as maximum likelihood estimation or morphological operators after manually initializing a point located on the femur. Other approaches use pattern recognition techniques with classifiers representing several image features developed using hundreds of training datasets.

As one example, in the case of fetal femur assessment known methods include morphological filtering wherein the image is first eroded with a large structuring element and the filtered image is subtracted from the original image to emphasize and segment the femur region. In another known semi-automatic method a user marks a point inside the femur region in the ultrasound image and the algorithm then utilizes a maximum likelihood framework to segment the entire femur. Yet another known approach for femur assessment is based on a training paradigm wherein a set of, for instance, 1000 images with labeled femurs, is used to train a probabilistic boosting tree. The parameters of the trained model are then used to estimate the femur length in test images. Still another known approach includes morphologically and computationally segmenting the femur.

Additionally, in the case of head circumference and biparietal diameter assessment, known methods include an automatic calculation by detecting inner and outer boundaries of a fetal skull using a computer vision technique known as active contour modeling. Another method is based on morphologically-based algorithms in order to recognize a fetal head contour in an ultrasound image, refine its shaped and compensate for irregularities, then measure its dimensions. In another method based on a learning approach, user annotated training data is obtained and classified via a discriminant classifier in a Probabilistic Boosting Tree.

Yet another approach is based on segmentation of fetal anatomic structures from echographic images. In this approach, contours of cranial cross-sections of fetal bodies are estimated and then measured. The contour estimation is formulated as a statistical estimation problem, where both the contour and the observation model parameters are unknown. The observation model relates, in probabilistic terms, the observed image with the underlying contour. This likelihood function is derived from a region-based statistical model, and the contour and observation model parameters are estimated according to a maximum likelihood criterion via deterministic iterative algorithms.

However, the above processes tend to be time-consuming, may include user intervention or a trained ultrasonographer, may be subject to operator variability, or may be prone to false detection. In remote or rural markets it may be particularly difficult to obtain services of a trained ultrasonographer or ultrasound technician, causing remote regions to be poorly served or underserved.

Therefore, it would be desirable to improve visualization techniques in ultrasound images in order to better estimate fetal gestational age and overcome the aforementioned drawbacks.

### BRIEF DESCRIPTION

Embodiments of the invention are directed to a method and apparatus for ultrasound imaging and, more specifically, automatically measuring fetal gestational age.

According to an aspect of the invention, an ultrasound system includes a transducer array comprising a multiplicity of transducer elements configured to acquire image data of an object, a display system for displaying an image of the object based on the acquired image data, and an image processor module. The image processor module is programmed to calculate the curvature of the image, and identify an object feature based on the calculated curvature and based on known feature tendencies of the object.

According to another aspect of the invention, a method a method of ultrasound image processing includes obtaining an image of at least a portion of a fetus, computing curvature at each point in the image, and computing an object feature based on the computed curvature and based on a known clinical feature of the fetus.

According to yet another aspect of the invention, a computer readable storage medium having stored thereon a computer program comprising instructions which when executed by a computer cause the computer to obtain an image of a fetus, calculate the curvature at points in the image, and calculate a feature of the fetus based on the calculated curvature and based on a feature of one or more fetuses obtained in another clinical setting.

These and other advantages and features will be more readily understood from the following detailed description of preferred embodiments of the invention that is provided in connection with the accompanying drawings.

### BRIEF DESCRIPTION OF THE DRAWINGS

FIG. 1 is a schematic block diagram of an ultrasound system according to an embodiment of the invention.

FIG. 2 illustrates a technique for determining gestational age, according to an embodiment of the invention.

FIG. 3 illustrates a technique for determining gestational age based on a measurement of a femur, according to an embodiment of the invention.

FIG. 4 illustrates a technique for determining gestational age based on a measurement of a cranium, according to an embodiment of the invention.

### DETAILED DESCRIPTION

According to embodiments of the invention, an ultrasound system is provided that functions to automatically detect and measure one of a femur and a cranium in a fetus and to automatically estimate gestational age of a fetus therefrom.

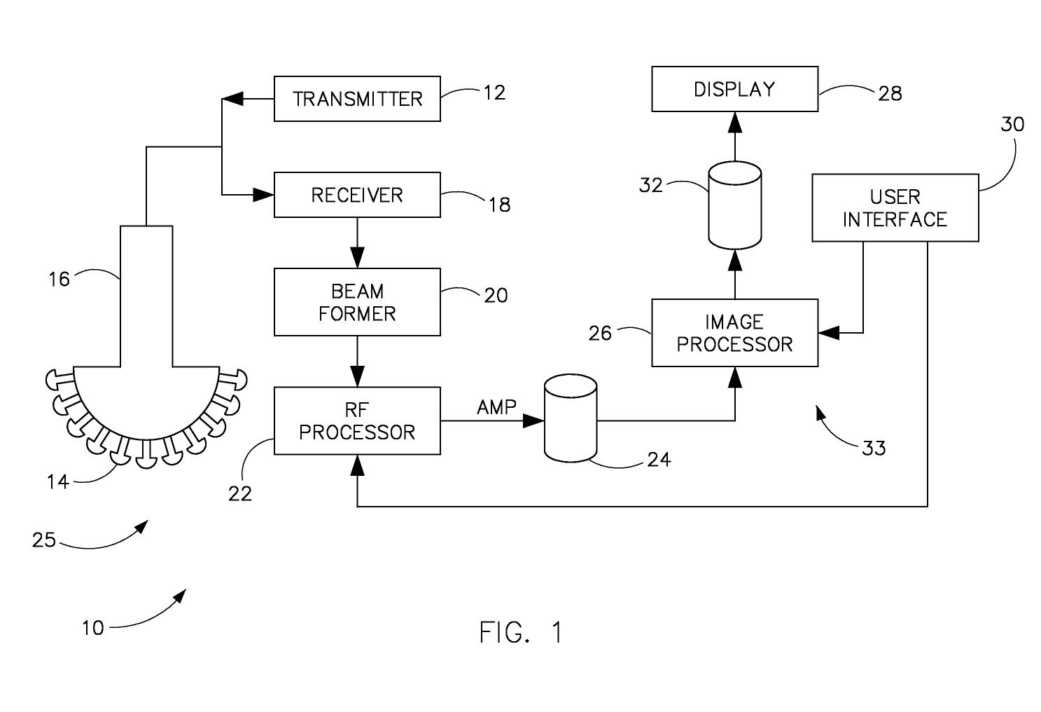

According to an embodiment of the invention, FIG. 1 illustrates an ultrasound system 10 including a transmitter 12 that drives an array of elements 14 (i.e., transducer elements) within an ultrasound transducer 16 to emit pulsed ultrasonic signals into a body or imaging volume. The elements 14 may be arranged, for example, in one or two dimensions. Each ultrasound transducer 16 has a defined center operating frequency and bandwidth. The ultrasonic signals are back-scattered from structures in the body, like fatty tissue or muscular tissue, to produce echoes that return to the elements 14. The echoes are received by a receiver 18 and are passed through beam-forming electronics 20 to acquire image data from the raw acoustic data received by ultrasound transducer 16. Beam-forming electronics 20 perform a beam-forming function and output an RF signal, which then passes through an RF processor 22. The RF processor 22 may include a complex demodulator (not shown) that demodulates the RF signal to form IQ data pairs representative of the echo signals. It may also include a gain and TGC/LGC control unit to adjust the signal amplitude. The RF signal or IQ data pairs may further be filtered, decimated, envelope detected, and compressed to form compressed envelope data. The image frame data sets (i.e., image data) are then routed to a memory 24 for storage or directly to an image processor module 26, according to embodiments of the invention. As shown in FIG. 1, the components 12-22 form front-end hardware 25.

According to embodiments of the invention, image processor module 26 is configured to process the acquired ultrasound information (i.e., image frame data sets) and prepare frames of ultrasound information for display on display 28. Acquired ultrasound information may be processed and displayed in real-time during a scanning session as the echo signals are received. Additionally or alternatively, the ultrasound information may be stored in memory 24 during a scanning session and then processed and displayed in an off-line operation.

The processor module 26 is connected to a user interface 30 that may control operation of the processor module 26. The display 28 includes one or more monitors that present patient information, including diagnostic ultrasound images to the user for diagnosis and analysis. One or both of memory 24 and memory 32 may store data sets of the ultrasound data, where such datasets are accessed to present 2D and 3D images. Multiple consecutive 3D datasets may also be acquired and stored over time, such as to provide real-time 3D or 4D display. The images may be modified and the display settings of the display 28 also manually adjusted using the user interface 30. As shown in FIG. 1, the components 24-32 collectively form back-end electronics 33.

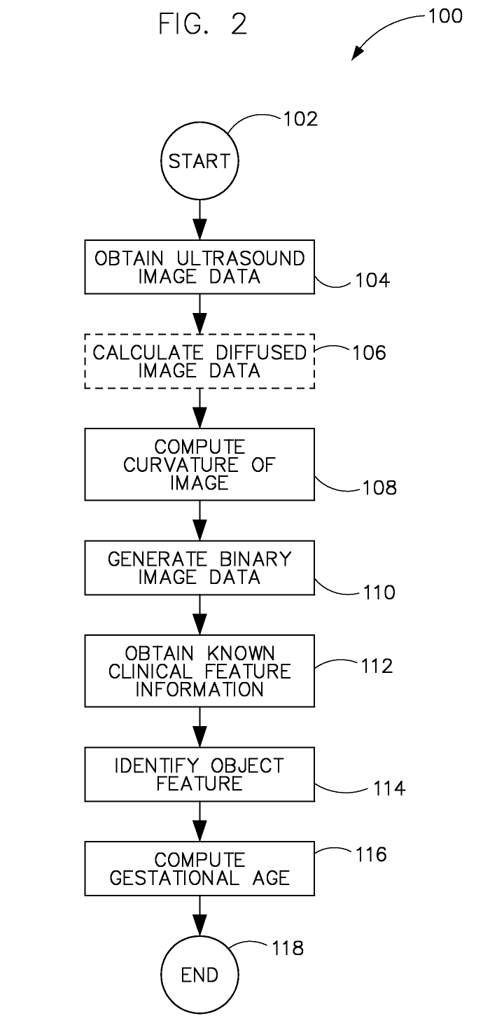

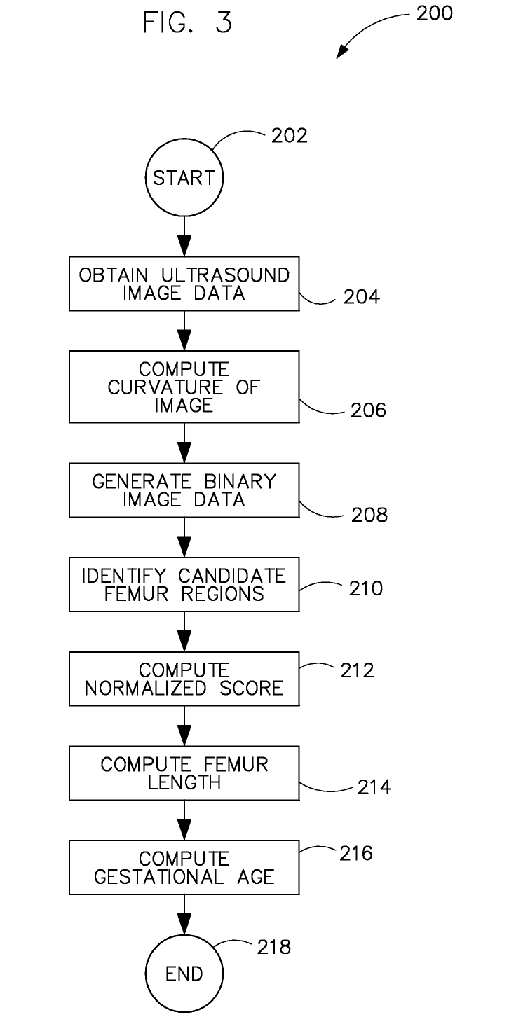

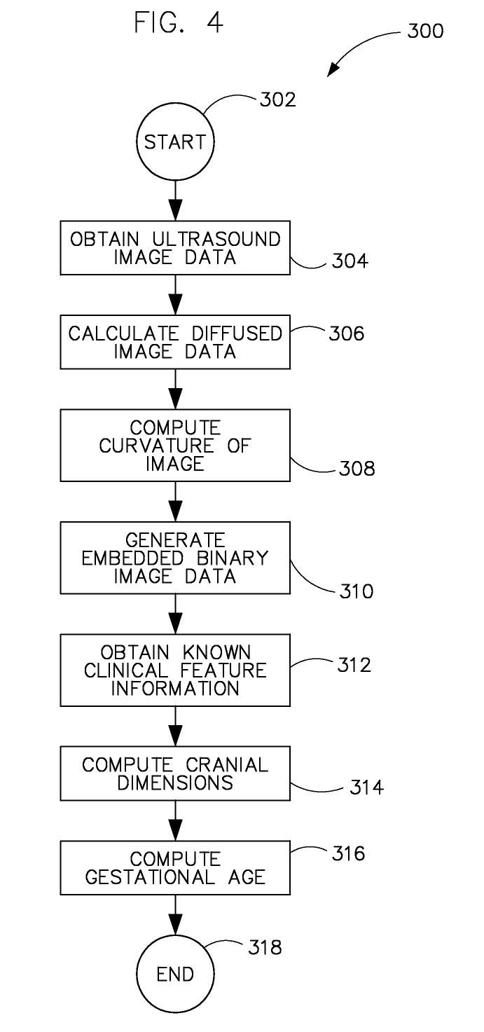

FIG. 2 illustrates a technique 100 for determining gestational age (GA), according to embodiments of the invention. Technique 100 includes but is not limited to calculating GA based on a determined femur length and based on a determined cranial diameter. Details more specific to calculation of femur length and GA determined therefrom will be illustrated with respect to FIG. 3, and details more specific to calculation of cranial diameter and GA determined therefrom will be illustrated with respect to FIG. 4.

Technique 100 includes a general technique for automatically identifying an object feature from ultrasound image data and computing GA therefrom. Technique 100 starts at step 102 and ultrasound image data is obtained at step 104. The ultrasound image data obtained at step 104 may include 2-D or 3-D ultrasound data. In embodiments of the invention, a diffusion operator is optionally applied to the ultrasound data to localize acoustically dense objects in the ultrasound image data.

The approach is premised on the knowledge that topology of noise in ultrasound images is more sensitive to diffusion than that of a physical object. Ultrasound imaging on structures (of size much greater than wavelength) with high characteristic impedance (such as bones) produce relatively high intensity specular echo signals. On the other hand, anatomical features that are small and/or have weak impedance either produce diffuse echoes or low intensity specular echoes that are subdued by surrounding noise. As a result, application of a diffusion operator can significantly alter the topology of regions in an ultrasound image characterized by diffusive and/or weak echoes. Because the topology in some regions shows greater variability in response to multi-level diffusion, the use of variance in topology as an outlier rejection may be used as an outlier rejection strategy to facilitate object detection in some applications, such as in fetal head detection, as a preprocessing step 106. As such, step 106 is optionally performed in imaging applications of regions sensitive to diffusion.

The curvature at each point on the input image is calculated at step 108. In one example, the curvature is calculated using the following equation:

$${curv}_{xy} = \frac{∇ \cdot ( \frac{\partial {I}_{xy}}{\partial x} \hat{x} + \frac{\partial {I}_{xy}}{\partial y} \hat{y} )}{\sqrt{{( \frac{\partial {I}_{xy}}{\partial x} )}^{2} + {( \frac{\partial {I}_{xy}}{\partial y} )}^{2}}} ; . \qquad Eqn . 1$$

where x, y refers to the pixel location co-ordinates. Once the curvature is calculated, image pixels above a threshold curvature are discarded (set to zero). In one embodiment, pixels having a curvature greater than −0.1 are discarded; however, it is to be understood that other thresholds may be used according to the invention and that other known methods for calculating a curvature may be employed.

In addition to the curvature threshold, all image pixels whose intensity is below a certain specified threshold are also discarded. In one embodiment, the intensity threshold is set based on an 8-bit integer range, and image pixels having an intensity that is less than half the 8-bit range are discarded or set to zero. In another embodiment the intensity threshold is automatically determined from the image data using automated techniques such as Otsu or K-means thresholding. Image pixels that were not discarded in either the curvature thresholding step or the intensity thresholding step are set to a high value such as one and binary image data or a binary image is generated at step 110. Further, it is to be understood that the invention is not limited to generation of binary data per se, but that any data may be generated and used that may be separated or otherwise binned into distinct datasets. For instance, data may be set to different colors or grayscales based on a given threshold.

Object features within the image or image data may be determined based on known clinical feature information or known feature tendencies of the object, as illustrated at step 112. For instance, clinical information obtained includes but is not limited to typical femur curvature or profile, or typical cranial shapes (such as an elliptical shape). Based on these profiles, components within the binary image or image data may be automatically selected, connected, and identified at step 114 to generate component combinations that best match the known clinical feature.

In one embodiment, step 114 includes selecting a minimum number of points (i.e., the inliers) to determine model parameters for matching to a known clinical feature, parameters of the model are solved. Find the number of points from the set of all points that fit with in a predefined tolerance and then add them to the inlier list. If the number of inliers exceeds a predefined threshold, then model parameters are re-estimated using identified inliers. Once complete, model parameters are appended and with associated cardinality to a model set, and these steps of step 114 are repeated as necessary. Models are down selected from a model set which includes parameters that are consistently found across a family of diffused images, at the same location with similar shape and size. Among the down selected models, the model having maximum cardinality is selected.

Once components are combined into a single object or a single object is identified, the object may then be automatically measured. As will be illustrated with respect to FIGS. 3 and 4, such objects may be a femur or a cranium, as examples, and the features measured therefrom or otherwise identified at step 114 may be, respectively, a femur length or a cranial diameter.

After identification of the object and determination of the feature(s) thereof at step 114, GA of the object or fetus may be determined at step 116, according to embodiments of the invention. Based on historical observation, such as femur length and cranial diameter as examples, measured data may be used to obtain GA therefrom as is understood in the art. Thus, it is to be understood that once an object feature is automatically identified and measured according to the invention, output may likewise be automatically determined based on historical data thereof. As such, GA may be automatically determined and presented based on object features and based on historical data, according to embodiments of the invention. Further, it is to be understood that technique 100, instead of or in addition to outputting GA at step 116, may output the object features that were computed at step 114. In such fashion a clinical expert may be employed or otherwise available to separately determine GA based on the automatically measured feature or features obtained from the ultrasound measurement. Technique 100 ends at step 118.

Thus, technique 100 finds instances of objects within a certain class of shapes that are found consistently across a family of images at a given location, and technique 100 is able to automatically compute GA therefrom. As illustrated with respect to FIGS. 3 and 4 below, it is possible to further refine numerical techniques to better classify and identify objects of interest for GA determination, according to embodiments of the invention. Such techniques include but are not limited to numerical weighting functions or other means of normalizing or scoring object data, or a regression technique.

Referring now to FIG. 3, a detection algorithm technique 200 is premised on the distribution and anatomical shape and presentation of a femur bone in typical fetal femur scans, and their sizes across the gestational trimesters. The femur is automatically detected from a 2-D or 3-D ultrasound image using a normalized score that accounts for a cumulative sum of several factors. Once the femur is localized, the measurement process utilizes a polynomial curve fitting technique to determine end-points of the bone from a 1-D profile that is, in one embodiment, most distal from a transducer surface.

Technique 200 begins at step 202 and 2-D or 3-D ultrasound imaging data is obtained at step 204. The method comprises automatic femur identification followed by automatic femur length measurement. The identification process involves automatic detection of candidate femur regions and selection of a single candidate femur region from all possible candidates. The automated measurement is made on the selected femur region, and may be implemented in numerical computing software such as Matlab®. Matlab is a registered trademark of The Mathworks, Inc., Delaware, Mass.

Candidate regions are obtained from an 8-bit grayscale image in one embodiment. At step 206 the curvature of image I as shown above in Equation 1 is computed, and image pixels having curvature greater than −0.1 are discarded, in one example. In addition, image pixels having an intensity less than half of an 8-bit integer range, as an example, are also discarded. At step 208 a binary image or image data is generated by setting the value of all the discarded pixels to zero and the setting the value of the remaining pixels to one. At step 210 candidate regions are obtained from the resulting binary image using, for instance, an 8-neighborhood connected component labeling. The femur is assumed to be bright and sharp-edged due to the high acoustic impedance of bone relative to surrounding soft tissue and an elongated structure located towards the center of the image display oriented at small angles to the probe surface. A five-parameter discriminator is used to compute a normalized score for each connected component at step 212.

The parameters of the five-parameter discriminator computed at step 212 are chosen on the basis of anatomy, tissue characterization in ultrasound, and scan geometry, as examples. These parameters include but are not limited to: (a) mean intensity (I); (b) aspect ratio (R); (c) distance of the centroid from the edges of the probe angle (D); (d) phase symmetry of the edge (φ); and (e) orientation or angle of the segment along the maximum dimension (θ).

The score is computed as follows:

$${S}_{i} = \frac{1}{5} \sum ( \begin{matrix}\frac{{\overline{I}}_{i}}{{max ( {\overline{I}}_{i} )}_{i = 1 , N}} + \frac{{\overline{R}}_{i}}{{max ( {\overline{R}}_{i} )}_{i = 1 , N}} + \frac{{\overline{D}}_{i}}{{max ( {\overline{D}}_{i} )}_{i = 1 , N}} + \\ \frac{{\overline{ϕ}}_{i}}{{max ( {\overline{ϕ}}_{i} )}_{i = 1 , N}} + \frac{{\overline{θ}}_{i}}{{max ( {\overline{θ}}_{i} )}_{i = 1 , N}}\end{matrix} ) ; . \qquad Eqn . 2$$

Following is further definition of the parameters of the five-parameter discriminator defined in Eqn. 2:

The first ratio includes a mean intensity parameter (I) and is computed by averaging the pixel intensity values corresponding to a candidate femur region.

Another potential discriminator is the aspect ratio (R) of the candidate femur regions with the femur bone exhibiting a smaller aspect ratio compared to other nearby structures. The aspect ratio of the candidate femur regions can be calculated as per the following steps: 1) image is segmented into four classes using an intensity-based multi-Otsu threshold as described by Liao, Chen, and Chung in A fast algorithm for multilevel thresholding, Journal of Information Science and Engineering, 17, 713-727 (2001) and all pixels not belonging to the brightest class are discarded; 2) the intensity of all the remaining image pixels is set to unity or 1; 3) the binary image is then divided into candidate regions based on connected component labeling; 4) the aspect ratio for each of the connected components is estimated based on the following procedure. The (p, q)^{th} central moment of a connected component is estimated as:

$${u}_{pq} = {\sum }_{x} {\sum }_{y} {{I}_{xy} ( x - \overline{x} )}^{p} {( y - \overline{y} )}^{q} ; , \qquad Eqn . 3$$

where, x, y is the set of pixel co-ordinates belonging to the connected region from step 2 and x̅, y̅ are the mean pixel-coordinates. Based on this expression the aspect ratio parameter can be computed as follows:

$$aspectRatioParam = 1 - \sqrt{\frac{{u}_{20} + {u}_{02} - \sqrt{4 {u}_{11}^{2} + {( {u}_{20} - {u}_{02} )}^{2}}}{{u}_{20} + {u}_{02} + \sqrt{4 {u}_{11}^{2} + {( {u}_{20} - {u}_{02} )}^{2}}}} ; , \qquad Eqn . 4$$

Finally, 4) the connected component regions from step 2 are matched with the candidate femur regions and the aspect ratio parameter is assigned (to the candidate femur region) based on the established correspondence.

The third ratio includes a distance parameter (D) and is computed by calculating a distance of a centroid (of the candidate femur region) to the closest edge corresponding to field of view of the ultrasound transducer.

The fourth ratio includes a phase parameter (φ) and is computed by calculating a median value of phase congruency values corresponding to the candidate femur region. The Fourier decomposition of an image gives rise to a magnitude signal and a phase signal. Based on the theory of phase congruency, the phase components are symmetric at the location of a step edge in the image. Because the femur bone presents very high impedance to an incident ultrasound beam, almost the entire incident beam is reflected back and the region below the femur is composed of dark intensity pixels giving rise to a sharp step edge in the image. Therefore the femur region is likely to exhibit a very high level of phase congruency at the surface distal to the transducer probe. This aspect is captured through the use of the phase congruency parameter as outlined.

The phase congruency value at each pixel location in the input image is calculated by employing a bank of Gabor filters at multiple scales. The convolution of the input image with the filter bank results in a series of complex valued outputs from which the phase congruency can be estimated, such as described by Kovesi in Symmetry and Asymmetry from Local Phase, at the 10^{th} Australian Joint Conference on Artificial Intelligence, 2-4 December, 1997. From the phase congruency image, the phase parameter (φ) is computed by calculating the median value of the phase congruency values corresponding to the candidate femur region.

The fifth ratio is an angle parameter (θ) and is calculated as follows:

$$orientation = \frac{1}{2} {tan}^{- 1} ( \frac{2 {u}_{11}}{{u}_{20} - {u}_{02}} ) ; , \qquad Eqn . 5$$

where u₁₁, u₂₀ and u₀₂ can be obtained from the candidate femur regions using Eqn. 3. Once the orientation of the candidate femur region is obtained, the angle parameter (θ) is computed as follows:

$$angleParam = \{ \begin{matrix}\sqrt{\frac{90 - orientation}{60}} & ∀ orientation \geq 30 ° \\ 1 & otherwise\end{matrix} ; \qquad Eqn . 6$$

Referring back to FIG. 3 and Eqn. 2, Si is the ith component out of a total of N connected components. The component that scores the maximum represents the femur region, is identified as such, and its length is automatically computed at step 214, according to an embodiment of the invention. Thus, at step 214 after the automatic selection of a region-of-interest, a 1-D profile of the femur most distal to the transducer is tracked by tracing rays from the bottom edge of the image along a vertical axis and fitting a polynomial curve with a least trimming square regression (LTS) method. End-points are determined from a discontinuity in the pattern of error values between actual coordinate and the point estimated by the LTS method. The cut-off is empirically established above the 90^{th} percentile of the sorted error value, as an example.

GA of the object or fetus may be determined based on the computed femur length at step 216 according to embodiments of the invention. It is to be understood that once the femur is automatically identified and measured according to the invention, output may likewise be automatically determined based on historical data thereof. As such, GA may be automatically determined and presented based on object features, according to embodiments of the invention. Further, it is to be understood that technique 200, instead of or in addition to outputting GA at step 216, may output the femur length computed at step 214. In such fashion a clinical expert may be employed or otherwise available to determine GA based on the automatically measured feature or features obtained from the ultrasound measurement. Technique 200 ends at step 218.

Referring now to FIG. 4, a detection algorithm technique 300 is illustrated that comprises automatic cranium identification followed by automatic cranium diameter measurement. Original image data is used to derive a family of images I(x,y,σ) that are obtained by convolving an original image I₀(x,y) with a Gaussian kernel G(x,y,σ) of variance σ:

I(x,y,σ)=I₀(x,y)*G(x,y,σ); Eqn. 7.

where Iₚₛ(x,y,σ) is a point set image containing a set of data points representing the image I(x,y,σ). As the image is subjected to noise, the data points include “inliers” that are data whose distribution can be explained by model or regressed parameters for fitting to an ellipse, as an example, and “outliers” which are data that do not fit the model. Hence, give a (typically small) set of inliers across multiple diffused images, parameters of the model are estimated that optimally describes this data. A regression technique is then used to fit the model, according to an embodiment of the invention.

The process begins at step 302 and 2-D or 3-D ultrasound imaging data is obtained at step 304. Diffused image data is calculated at step 306 by subjecting the original image I₀(x,y) to a diffusion operator to generate a family of images I(x,y,σ).

To extract features a divergence of a gradient vector field is calculated at step 308 in order to calculate a curvature of the image, as described above with respect to Eqn. 1. The relevant gradient field for this image is the vector field depicting the rate of change of intensity at each point. If a point is on a cranium, then it will have intensity higher than its neighbor such that the vector field points inward towards that region. Therefore, the divergence of the vector field in that region would have a negative value, and the region is referred to as a sink. If the region does not belong to cranium, then the divergence is typically positive and the region is referred to as a source.

Although the cranium tends to be high intensity, there also tends to be considerable inconsistency across its structure. The variation may be caused by differences in acoustic impedances based on alignment of the structure, or variation may be introduced by different users, changing scan parameters, and the like. Thus, structural and intensity information is combined to form an embedded binary image at step 310 as described below.

The diffused image data is multi-level thresholded to capture intensity information, where φ_{I}^{k} is the value of the kth threshold level. A connected component I_{cc}(x,y) is generated with the lowest k value (k=lower):

$${I}_{cc} ( x , y ) = \{ \begin{matrix}1 & if {I}_{F} ( x , y ) < {φ}_{{I}_{F}} and I ( x , y ) > {φ}_{I}^{lower} \\ 0 & else\end{matrix} ; , \qquad Eqn . 8$$

where φ_{I}_{_{F}}, and φ_{I} are respective thresholds on I_{F} and I. The I_{F}(x,y) set is less than φ_{I}_{_{F}} as the region with negative values represents the cranium. The cranium is a positive intensity region, thus I(x,y) is a set greater than φ_{I}^{lower}. The connected component in the image I_{cc} is labeled with labels associated to intensity levels. In one example a two-level threshold is selected. In this example, dᵢ^{l} is the ith connected component (Ωᵢ) with label l in the image I_{cc} such that:

$$l = \{ \begin{matrix}lower if I ( x , y ) < {φ}_{I}^{upper} & ∀ ( x , y ) ∈ {Ω}_{i} \\ upper if I ( x , y ) \geq {φ}_{I}^{upper} & ∃ ( x , y ) ∈ {Ω}_{i}\end{matrix} ; . \qquad Eqn . 9$$

The multi-level labeling is possible because a high intensity region, always contained in a low intensity region (i.e. having an inclusive threshold), thus a tree structure may be formed. Further, labeling based on intensity can be seen as embedding contrast information on connected component image I_{cc}.

At step 312 known clinical information is obtained, such as in the case of a cranial measurement, best fit parameters for an elliptical shape and known clinical parameters from the imaging protocol. At step 314 cranial dimensions are obtained. In one example the cranial dimensions are obtained according to the following steps:

The input image which is composed of Q connected components is indicated by D={d₁^{l}, . . . , d_{Q}^{l}} where label lε(lower,upper) and a Minimal sample set (MSS is a set, which at least contains the minimal number of points required to uniquely fit a model) may be indicated with a letter s. Let φ({d₁^{l}, . . . , d_{Q}^{l}}) be a parameter vector estimated using the set of data {d₁^{l}, . . . , d_{Q}^{l}}, where h≧k and k is the cardinality of the MSS. Invariably a connected component has at least four points that are necessary and sufficient conditions to draw a unique ellipse. Hence, k is set to one. As the cranium has high intensity, at least one connected component in the MSS should have the label l=upper. The model M is defined as M(φ)≡{dεR^{d}:ƒ_{M}(d;φ)=0, where φ is a parameter vector and ƒ_{M} is a function containing all the points that fit the model M instantiated with the parameter vector φ. The error associated with the datum d is defined with respect to the manifold M(φ) as a distance from do to M(φ), where

$$e ( d , M ( ϕ ) ) ≡ \frac{1}{N} {min}_{{d}^{′} ∈ M ( ϕ )} dist ( d , {d}^{′} ) ,$$

and where dist(•,•) is an appropriate distance function and N the normalizing factor is the number of points in d. For an ellipse, a least square fitting to ellipses may be determined, as understood within the art, to generate an error metric, such as Fitzgibbon's as defined in Direct Least Square Fitting of Ellipses, IEEE Trans. Pattern Anal., Mach. Intell., vol. 21, No. 5, pp. 476-480, 1999. Using this error metric, the fetal head characteristics (such as size and shape), and clues from the clinical protocol, CS is defined as:

S(φ)≡{dεD:e(d,M(φ))≦p(φ)≦pₘₐₓ,

η(φ)≦ηₘₐₓ,∠(φ)∠≦∠ₘₐₓ}; Eqn. 10

where δ is a threshold on a cost of ellipse fit, which is inferred from the nature of the problem. (pmax, pmin), ηₘₐₓ and Φₘₐₓ are bounds on the perimeter p, eccentricity η and angle of inclination Φ, respectively. Perimeter and eccentricity bounds may be extracted, for instance, using a Hadlock table as defined in Estimating fetal age: Computer assisted analysis of multiple fetal growth parameters, Radiology, vol. 152, pp. 497-501, 1984. The limit on angle inclination is set based on clinical guidelines. The variance of parameter (VoP) is computed using only the elements that are in parameter space φ and are consistent across all diffused images I_{σ}_{_{K}}:

var(\hat{φ}/Sᵢ)≡{E{( φ̅−\hat{φ}ⱼ)²}:( φ̅−\hat{φ}ⱼ)<φ_{(VoP)},

φⱼεSⱼ

(∃Sⱼ)ε(∀I_{σ}_{_{K}})}; Eqn. 11

where φ_{(VoP)} is the size of the accumulator grid in parameter space.

The diffusion based regression algorithm thus includes three steps: 1) Minimal sample sets (MSSs) are randomly selected from the input dataset and the model parameters are computed using only the elements of the MSS. The cardinality of the MSS is the smallest and sufficient to determine the model parameters (e.g., if the model is a line or ellipse then the cardinality should be at least two or four, respectively). 2) Check which elements of the dataset are consistent with the model instantiated with the parameters estimated in the first step (The set of such elements is called a consensus set—CS). 3) Find instances of objects within a certain class of shapes that are found consistently across the family of images at the same location by a voting procedure. This voting procedure is carried out in a parameter space, from which object candidates are obtained as local maxima in a so-called accumulator space (that is explicitly constructed by the algorithm for computing a Hough transform), given that the local maxima contains candidates from all the diffused images. The grid size of the accumulator space is fixed based on certain threshold on the variance of parameters (VoP) which is used to put a cap on variability of the topology. Finally, the algorithm terminates when the probability of finding a better ranked CS amongst the candidate. Once the CS is localized, an ellipse fitting technique, such as described in Fitzgibbon above, is used to draw an ellipse. The cardinality of the CS in case of an elliptical model is estimated as the number of discrete points of the fitted ellipse that lie on the object. The points on the ellipse circumference are discretized based on constant angular span. The angular discretization of the ellipse circumference, has a normalizing effect on the cardinality, across various scales of ellipses. In embodiments of the invention, this ellipse may be further refined based on imaging statistics, as understood in the art.

For instance, an ellipse fitting energy can be formulated, composing of a region-based term using Gaussian statistics, and a feature based term that pulls the ellipse away from local minima, towards extracted cranium pixels referred previously by the binary image I_{cc}. The motivation of the feature-based term is that pixels corresponding to cranium structures have a high mean curvature, a high intensity, and are of a higher scale when compared to noise and artifacts.

The following steps are devised for the energy formulation:
1. Let E denote the feature set region given by the binary image I_{cc}.
2. Let I: Ω→R be the image. Decompose Ω into K disjoint rectangles, such that Ω=Uᵢ₌₁^{K}Bᵢ, where the intensity in each of the rectangles is modeled using a bi-modal Gaussian distribution.
3. Let Cₒ^{d} and C_{bg}^{d} denote respectively the object region (region inside of the cranium) and the background region that lies within a distance d from the ellipse C.
4. μ^{o}=[μ₁^{o}, μ₂^{o}, . . . , μ_{K}^{o}] and σ^{o}=[σ₁^{o}, σ₂^{o}, . . . σ_{K}^{o}] represent respectively the vector of mean and variance parameters characterizing the Gaussian distribution in the regions Cₒ^{d}∩Bᵢ, and μ^{bg},σ^{bg} distribution corresponding to the region C_{bg}^{d}∩Bᵢ.
5. Minimization of the following energy functional over a parameterized ellipse C:[0,1]→Ω, with ellipse parameters (a, b, θ, c₀) yields an optimal (elliptical) fit for the cranium boundary.

$$J [ ( a , b , θ , {c}_{0} ) , ( {μ}^{o} , {σ}^{o} ) , ( {μ}^{bg} , {σ}^{bg} ) ] = {\sum }_{i = 1}^{K} {\int }_{{C}_{o}^{d}} {χ}_{{B}_{i}} \{ \frac{{( I - {μ}_{i}^{o} )}^{2}}{{σ}_{i}^{{o}^{2}}} - ln ( {σ}_{i}^{o} ) \} d x + {\int }_{{C}_{b}^{d}} {χ}_{{B}_{i}} \{ \frac{{( I - {μ}_{i}^{bg} )}^{2}}{{σ}_{i}^{{bg}^{2}}} - ln ( {σ}_{i}^{bg} ) \} d x + λ {\int }_{C} {d}_{E}^{2} d t + {w ( a - η b )}^{2} ; , \qquad Eqn . 12$$

Given the estimates μ^{o},σ^{o},β^{bg},σ^{bg}, the first and second terms of the energy drives the ellipse C to partition the rectangle Bᵢ into two regions; Cₒ^{d}∩Bᵢ and C_{b}^{d}∩Bᵢ where the distribution is close to (μ^{o},σ^{o}) and (μᵢ^{bg},σᵢ^{bg}). The third term pulls the ellipse to the boundary of feature set E and towards high intensity regions, governed by parameter λ. The feature-based weight

$${d}_{E} = {D}_{E} + κ \frac{1}{1 + τ I} ,$$

where D_{E} is the distance function to the feature set E, the intensity-based term

$$\frac{1}{1 + τ I}$$

drives C towards high intensity regions. κ, τ are tunable parameters that balance the feature term and the intensity term. The last term controls the eccentricity of the ellipse, where η is the eccentricity of the ellipse.
6. To minimize J, we use steepest descent to iteratively solve for (μ^{o},σ^{o},μ^{bg},σ^{bg}) and ellipse C, given an initial guess C^{o}. Since only rectangles Bᵢ that intersect with C affect the update equations, we compute distributions only at such rectangles. To optimize the number of rectangles used, and for better distribution estimates, rectangles are centered at Cₖⁿ=[xₖⁿ, yₖⁿ], where xₖⁿ,yₖⁿ are discrete ellipse points at the n^{th} iteration step.

The above framework can also be extended for exact segmentation of a cranial region for thickness measurements, as an example, the previous energy can be modified to search for two smooth curves, lying on the inside and outside boundaries of a cranial region. Also, to handle missing or low contrast boundaries, and shading effects close to the boundary, smoothness constraints of the generated curves and width constraints between the curves can be incorporated within the energy.GA of the object or fetus may be determined based on the computed cranial dimensions at step 316, according to embodiments of the invention. It is to be understood that once the cranium is automatically identified and measured according to the invention, output may likewise be automatically determined based on historical data thereof. As such, GA may be automatically determined and presented based on object features, according to embodiments of the invention. Further, it is to be understood that technique 300, instead of or in addition to outputting GA at step 316, may output the cranial dimensions computed at step 314. In such fashion a clinical expert may be employed or otherwise available to determine GA based on the automatically measured feature or features obtained from the ultrasound measurement. Technique 300 ends at step 318.

An implementation of embodiments of the invention in an example comprises a plurality of components such as one or more of electronic components, hardware components, and/or computer software components. A number of such components can be combined or divided in an implementation of the embodiments of the invention. An exemplary component of an implementation of the embodiments of the invention employs and/or comprises a set and/or series of computer instructions written in or implemented with any of a number of programming languages, as will be appreciated by those skilled in the art.

An implementation of the embodiments of the invention in an example employs one or more tangible computer readable storage media. An example of a computer-readable storage medium for an implementation of embodiments of the invention comprises the recordable data storage medium of the image reconstructor 34, and/or the mass storage device 38 of the computer 36. A computer-readable storage medium for an implementation of embodiments of the invention in an example comprises one or more of a magnetic, electrical, optical, biological, and/or atomic data storage medium. For example, an implementation of the computer-readable signal-bearing medium comprises floppy disks, magnetic tapes, CD-ROMs, DVD-ROMs, hard disk drives, and/or electronic memory.

A technical contribution for the disclosed method and apparatus is that it provides for a computer-implemented apparatus and method of automatically assessing human gestational age.

According to an embodiment of the invention, an ultrasound system includes a transducer array comprising a multiplicity of transducer elements configured to acquire image data of an object, a display system for displaying an image of the object based on the acquired image data, and an image processor module. The image processor module is programmed to calculate the curvature of the image, and identify an object feature based on the calculated curvature and based on known feature tendencies of the object.

According to another embodiment of the invention, a method of ultrasound image processing includes obtaining an image of at least a portion of a fetus, computing curvature at each point in the image, and computing an object feature based on the computed curvature and based on a known clinical feature of the fetus.

According to yet another embodiment of the invention, a computer readable storage medium having stored thereon a computer program comprising instructions which when executed by a computer cause the computer to obtain an image of a fetus, calculate the curvature at points in the image, and calculate a feature of the fetus based on the calculated curvature and based on a feature of one or more fetuses obtained in another clinical setting.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Furthermore, while single energy and dual-energy techniques are discussed above, the invention encompasses approaches with more than two energies. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. An ultrasound system comprising:
a transducer array comprising a multiplicity of transducer elements configured to acquire image data of an object;
a display system for displaying an image of the object based on the acquired image data; and
an image processor module programmed to:
calculate the curvature of the image; and
identify an object feature based on the calculated curvature and based on known feature tendencies of the object.

2. The system of claim 1 wherein the image processor module is programmed to diffuse the image prior to calculating the curvature of the image.

3. The system of claim 1 wherein the object feature is one of an object length and an object size.

4. The system of claim 1 wherein the image processor module is programmed to calculate binary image data based on the calculated divergence of the gradient vector field.

5. The system of claim 4 wherein the image processor module is programmed to calculate the one of the object length and the object diameter based on the calculated binary image data.

6. The system of claim 1 wherein the object is one of a femur and a cranium within a fetus.

7. The system of claim 1 wherein the image processor module is programmed to calculate a normalized score based on a plurality of imaging parameters and identify the object as a femur based on the normalized score.

8. The system of claim 7 wherein the plurality of imaging parameters comprises a mean intensity of the object, an aspect ratio of the object, a distance of a centroid of the object from edges of a probe angle, a phase symmetry of an edge of the object, and an orientation segment along a maximum dimension of the object.

9. The system of claim 1 wherein the known feature tendencies comprise at least one of a polynomial shape and an elliptical shape.

10. A method of ultrasound image processing, the method comprising:
obtaining an image of at least a portion of a fetus;
computing curvature at each point in the image; and
computing an object feature based on the computed curvature and based on a known clinical feature of the fetus.

11. The method of claim 10 further comprising diffusing the image prior to computing the curvature of the image.

12. The method of claim 10 wherein computing the object feature comprises one of an object length and an object diameter.

13. The method of claim 10 further comprising generating a binary image of the object and wherein computing the object feature comprises computing the object feature based on the binary image.

14. The method of claim 10 wherein obtaining the image of at least a portion of the fetus comprises obtaining an image of one of a femur and a cranium.

15. The method of claim 10 further comprising calculating a normalized score for two or more connected components within the image and identifying the two or more connected components as a femur based on the normalized score.

16. The method of claim 15 wherein the known clinical features of the fetus include a curvature of the femur.

17. A computer readable storage medium having stored thereon a computer program comprising instructions which when executed by a computer cause the computer to:
obtain an image of a fetus;
calculate the curvature at points in the image; and
calculate a feature of the fetus based on the calculated curvature and based on a feature of one or more fetuses obtained in another clinical setting.

18. The computer readable storage medium of claim 17 wherein the computer is further caused to calculate diffused image data from the obtained image data prior to calculating the curvature of the image.

19. The computer readable storage medium of claim 17 wherein the computer is caused to generate a binary image of the fetus from the obtained image.

20. The computer readable storage medium of claim 17 wherein the computer is caused to calculate the feature of the fetus to include one of a femur curvature and an elliptical shape of a cranium.
